(19) United States
(12) Patent Application Publication
(10) Pub. No.: US 2013/0143821 A1
(43) Pub. Date: Jun. 6, 2013

(54) HYDROPHOBICALLY-MODIFIED HYALURONAN AND METHODS OF MAKING AND USING THEREOF

(71) Applicants: Jules John Magda, Salt Lake City, UT (US); Grant D. mith, Salt Lake City, UT (US); Dmitry Bedrov, Salt Lake City, UT (US); Jimmy W. Mays, Knoxville, TN (US)

(72) Inventors: Jules John Magda, Salt Lake City, UT (US); Grant D. mith, Salt Lake City, UT (US); Dmitry Bedrov, Salt Lake City, UT (US); Jimmy W. Mays, Knoxville, TN (US)

(73) Assignees: UNIVERSITY OF TENNESSEE RESEARCH FOUNDATION, Knoxville, TN (US); UNIVERSITY OF UTAH RESEARCH FOUNDATION, Salt Lake City, UT (US)

(21) Appl. No.: 13/576,994

(22) PCT Filed: Feb. 3, 2011
(86) PCT No.: PCT/US11/23544

§ 371 Date: Feb. 21, 2013

Related U.S. Application Data
(60) Provisional application No. 61/301,301, filed on Feb. 4, 2010.

(51) Int. Cl. A61K 38/14

(52) U.S. Cl. CPC A61K 38/14
USPC 514/16.7; 530/322; 514/20.9; 514/18.8

(57) ABSTRACT

Described herein are modified hyaluronans or the pharmaceutically-acceptable salt or ester thereof, wherein the modified hyaluronan comprises at least one hydrophobic polypeptide covalently bonded to hyaluronan. The modified hyaluronans can be used as viscosupplements in a number of medical applications. The modified hyaluronans can also be used in several biological and medical applications. Methods for preparing the modified hyaluronans are also provided herein.

### CROSS REFERENCE TO RELATED APPLICATIONS

This application claims priority upon U.S. provisional application Ser. No. 61/301,301, filed Feb. 4, 2010. This application is hereby incorporated by reference in its entirety.

### ACKNOWLEDGEMENTS

The research leading to this invention was funded in part by a National Institutes of Health (NIH) Grant No. R21EB004947. The U.S. Government has certain rights in this invention.

### BACKGROUND

Hyaluronan is a linear polysaccharide that is present in all living subjects. In vivo, hyaluronan has a molecular weight of 4-10M Da. Hyaluronan is present in the synovial fluid, which lubricates and cushions joints. However, hyaluronan degrades in arthritic joints. What is needed is a form of hyaluronan that is easy to handle and administer to a subject. Preferably, the mode of administration should be fairly non-invasive. It is also desirable that the hyaluronan be able to carry and deliver therapeutic agents useful in the treatment of a number of medical and biological applications. The modified hyaluronans described herein address these needs.

### SUMMARY

Described herein are modified hyaluronans or the pharmaceutically-acceptable salt or ester thereof, wherein the modified hyaluronan comprises at least one hydrophobic polypeptide covalently bonded to hyaluronan. The modified hyaluronans can be used as viscosupplements in a number of medical applications. The modified hyaluronans can also be used in several biological and medical applications. Methods for preparing the modified hyaluronans are also provided herein. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows the synthesis (top) and living polymerization (bottom) of amino acid anhydride L-leucine NCA.

FIG. 2 shows the grafting of poly(leucine) chains to linear HA chains (tertiary butylamine salts) at the hydroxyl group of glucosamine, modified into a vinyl group (step not shown).

FIG. 3 shows the comparison of poly(leucine) HA and a universal plot for zero-shear-rate limiting viscosity of solutions of unmodified HA of various molecular weights and concentrations, plotting specific viscosity as a function of overlap factor. [symbol] leucine-modified HA sample A5, x poly(leucine) modified HA sample B1, □ unmodified 132 kDa HA, Δ unmodified 67 kDa HA, ∘ unmodified 1500 kDa HA, literature experimental values, [symbol] empirical correlation of a universal plot for zero-shear-rate limiting viscosity.

FIG. 4 shows the creep-recovery behavior of sample A5 and 132 k Da unmodified HA at 5 wt %; a constant stress of 0.2 Pa was applied at time equal zero and removed at 60 s:. □ unmodified HA 132 k HA, [symbol] leucine-modified HA sample A5.

FIG. 5 shows the creep-recovery of 67 kDa unmodified HA at 5 wt % concentration, where a constant stress of 0.2 Pa was applied at time equal zero and removed at 60 s.

FIG. 6 shows the creep-recovery behavior of leucine-modified HA sample B1 at 5 wt %, where a constant stress of 0.2 Pa was applied at time equal zero and removed at 60 s.

FIG. 7 shows creep-recovery of leucine-modified B1 at 7.5 wt %, where a constant stress of 0.2 Pa was applied at time equal zero and removed at 60 s. The virtual absence of creep in the first 60 s indicated true gel behavior.

FIG. 8 shows the logarithmic plot of the elastic modulus G′ vs. oscillatory shear frequency of leucine-modified HA of various concentrations. (+) 7.5% B1, (x) 5% B1 and ( [symbol] ) 5% A5.

FIG. 9 shows the viscosity vs. shear rate for (□)132 kDa unmodified HA and ( [symbol] ) leucine-modified sample A5 at 5 wt %.

FIG. 10 shows the viscosity vs. shear rate for (□)132 kDa unmodified HA and ( [symbol] ) leucine-modified sample A5 at 3 wt % concentration.

FIG. 11 shows the viscosity vs. shear rate for (4) 67 kDa unmodified HA and (x) leucine-modified HA sample B1 at 5 wt % concentration.

FIG. 12 shows viscosity vs. shear rate for (X) leucine-modified HA sample B1 at 7.5 wt % concentration and (Δ) unmodified 67 k Da HA at 7.5 wt %. The B1 results show evidence of thixotropy.

FIG. 13 shows the surface tensions of 67 k Da unmodified HA at 0.05 mg/ml concentration (Δ) and B1 at 0.05 mg/ml (▴) and 1 mg/ml( [symbol] ).

FIG. 14 shows the surface tension of 132 k Da unmodified HA at 0.05 mg/ml (□) and 1 mg/ml(□) and A5 at 0.05 mg/ml (▪) and 1 mg/ml( [symbol] ).

FIG. 15 shows the equilibrium surface tensions of modified (solid datapoints) and unmodified (hollow datapoints) HA. 67 k Da unmodified HA(Δ), B1( [symbol] ), unmodified 132 k Da HA(□) and A5(▪).

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a macromolecule” includes mixtures of two or more such macromolecules, and the like.

“Optional” or “optionally” means that the subsequently described event or circumstance can or cannot occur, and that the description includes instances where the event or circumstance occurs and instances where it does not. For example, the phrase “optional targeting group” means that the group may or may not be present in the modified hyaluronan.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. For example, hyaluronan that contains at least one —OH group can be represented by the formula Z—OH, where Z is the remainder (i.e., residue) of the hyaluronan molecule.

Variables such as R¹, L, Y, and Z used throughout the application are the same variables as previously defined unless stated to the contrary.

### I. MODIFIED HYALURONAN AND PREPARATION THEREOF

Described herein are modified hyaluronans or the pharmaceutically-acceptable salts or esters thereof, wherein the modified hyaluronan comprises at least one hydrophobic polypeptide covalently bonded to hyaluronan. Due to the presence of the hydrophobic polypeptide, the modified hyaluronans described herein have unique rheological properties. As will be discussed in greater detail below, the modified hyaluronans have numerous biological and medical applications in view of their enhanced rheological properties.

Hyaluronan is a non-sulfated glycosaminoglycan (GAG). Hyaluronan is a well known, naturally occurring, water soluble polysaccharide composed of two alternatively linked sugars, D-glucuronic acid and N-acetylglucosamine. The polymer is hydrophilic and highly viscous in aqueous solution at relatively low solute concentrations. It often occurs naturally as the sodium salt, sodium hyaluronate. Methods of preparing commercially available hyaluronan and salts thereof are well known. Hyaluronan can be purchased from Seikagaku Company, Novozymes Biopolymers, Inc., LifeCore, Inc., Hyalose, Inc., Genzyme, Inc., Pharmacia Inc., Sigma Inc., and many other suppliers. In one aspect, the lower limit of the molecular weight of the hyaluronan useful herein is from 50,000 Da, 60,000 Da, 70,000 Da, 80,000 Da, 90,000 Da, or 100,000 Da, and the upper limit is 200,000 Da, 300,000 Da, 400,000 Da, 500,000 Da, 600,000 Da, 700,000 Da, 800,000 Da, 900,000 Da, 1,000,000 Da, 2,000,000 Da, 4,000,000 Da, 6,000,000 Da, 8,000,000 Da, or 10,000,000 Da where any of the lower limits can be combined with any of the upper limits. In one aspect, the hyaluronan has a molecular weight of 50,000 to 3,000,000.

The modified hyaluronan has at least one hydrophobic polypeptide covalently bonded to hyaluronan. The term “hydrophobic polypeptide” is defined herein as any polypeptide that exhibits the “hydrophobic effect,” which is the tendency of the polypeptide to aggregate with like molecules in water.

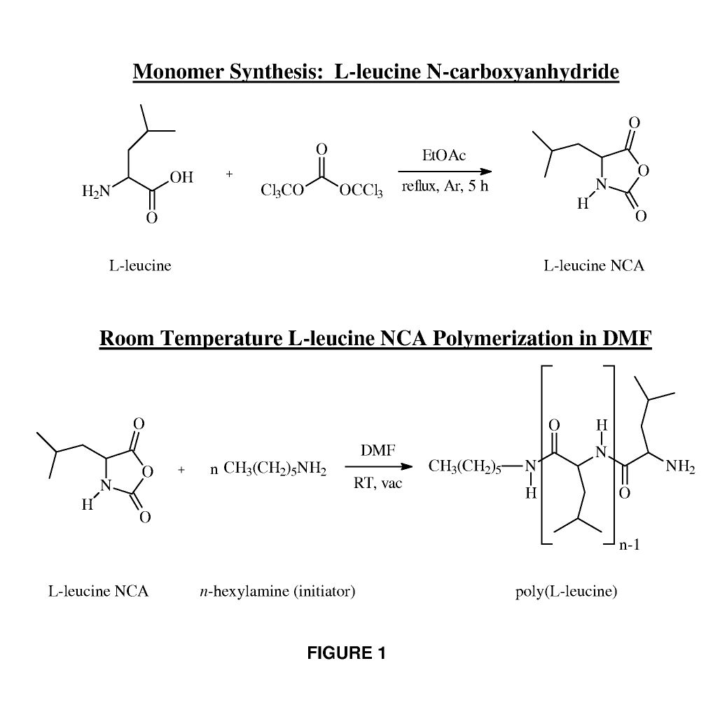

The polypeptide can be composed of a variety of different amino acid residues. In one aspect, the polypeptide comprises one or more leucine residues. In another aspect, the polypeptide is poly(leucine). In one aspect, the hydrophobic polypeptide is isotactic poly(leucine), atactic poly(leucine), or a combination thereof. Methods for making the different types of poly(leucines) are known in the art (see H. R. Kricheldorf and D. Muller, Macromolecules 16, 615-623 (1983); J. A. Hanson et al., Nature 455, 85-89 (2008); and B. Hanson et al., European Polymer Journal 46, 2310-2320 (2010)). Exemplary procedures for producing hydrophobic polypeptides useful herein are provided in FIG. 1 and in the Examples.

In one aspect, the hydrophobic polypeptide has 5, 6, 7, 8, 9, 10, 11, 12, 13, 14, 15, 16, 17, 18, 19, 20 amino acid residues, or any range thereof. In another aspect, the hydrophobic polypeptide has a molecular weight of 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000 or any range thereof.

The number of hydrophobic polypeptides that be can bonded to hyaluronan can vary as well. In one aspect, the modified hyaluronan has a grafting ratio of 1%, 2%, 3%, 4%, 5%, 6%, 7%, 8%, 9%, 10% of the HA repeat units. The term “grafting ratio” is defined as the number of hydrophobic polypeptide chains per repeat unit in hyaluronan [(GlcNAc-GlcUA-)x].

The hydrophobic polypeptide can be covalently bonded to the hyaluronan by a variety of techniques. In one aspect, the polypeptide can be directly bonded to the hyaluronan. For example, a hydroxyl group present in hyaluronan can react directly with a group present on the polypeptide (e.g., a carboxyl group) to form a new covalent bond. In another aspect, the polypeptide can be bonded to the hyaluronan via a linker. For example, the modified hyaluronan comprises a residue of formula I

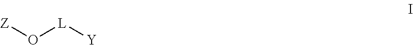

wherein
Z is a residue of hyaluronan;
L is a linker; and
Y is a residue of a hydrophobic polypeptide.

The nature of the linker can vary. In one aspect, the linker can be a second polypeptide that is not a hydrophobic peptide. For example, the hydrophobic polypeptide comprises diblock polypeptide comprising a hydrophilic block and a hydrophobic block, wherein the hydrophilic block is covalently attached to hyaluronan. In this aspect, the hydrophilic block is the linker. For example, the hydrophilic block can be poly(lysine) or poly(glutamate). In one aspect, the hydrophobic block is poly(leucine).

In other aspects, the linker can be any organic group that (1) can form a covalent bond with the hyaluronan and (2) has at least one functional group that can react with the hydrophobic polypeptide to form a covalent bond between the linker and the hydrophobic polypeptide. For example, the linker can be an organic group having at least one group capable of undergoing a Michael addition with the hydrophobic polypeptide. Examples of such groups include α,β-unsaturated carbonyl compounds such as, for example, esters, ketones, aldehydes, and anhydrides. In one aspect, the linker comprises an acrylate or methacrylate. In this aspect, the modified hyaluronan comprises a residue of formula II

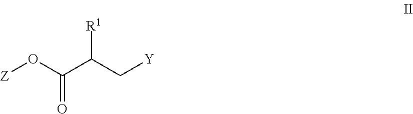

wherein
Z is a residue of hyaluronan;
R¹ is hydrogen or methyl; and
Y is a residue of a hydrophobic polypeptide.

In one aspect, the modified hyaluronan has a residue of formula II, wherein R¹ is hydrogen and Y is poly(leucine).

Methods for making the modified hyaluronans are also described herein. In one aspect, the modified hyaluronan is produced by the process comprising
(a) reacting hyaluronan with a linker to produce a covalent bond between the linker and hyaluronan, wherein the linker possesses at least one group capable of reacting with an amino acid residue present in a hydrophobic polypeptide to produce a covalent bond between the linker and the hydrophobic polypeptide; and
(b) reacting the hyaluronan produced in step (a) with the hydrophobic polypeptide.

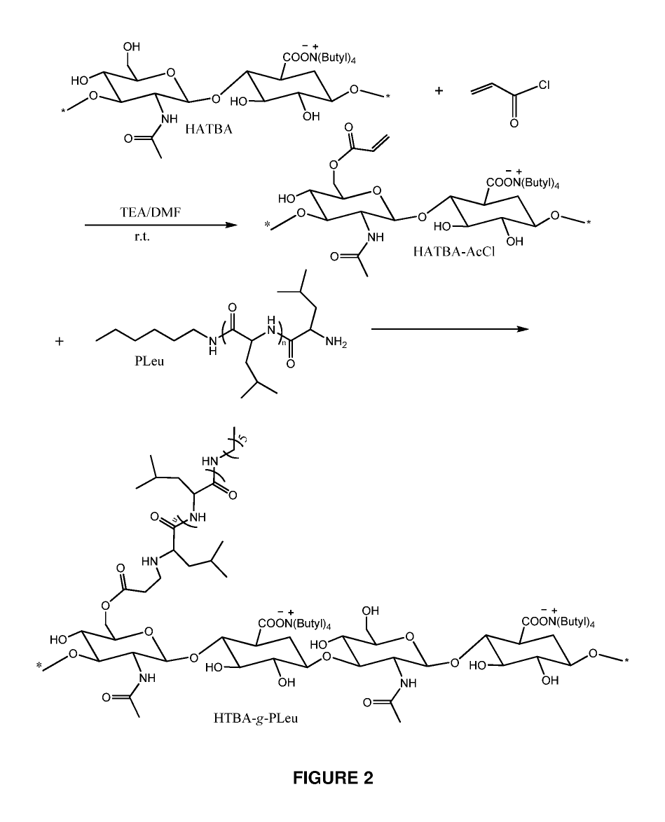

An exemplary procedure for producing modified hyaluronan using an acrylate as the linker to link poly(leucine) to hyaluronan is provided in FIG. 2. Additionally, exemplary synthetic procedures for making modified hyaluronans are provided in the Examples.

In certain aspects, the modified hyaluronan has a targeting group covalently bonded to the hydrophobic polypeptide. In this aspect, the targeting group can be useful in the adhesion and/or delivery of the modified hyaluronan into cells. The targeting agent can be a protein, peptide, an antibody, an antibody fragment, one of their derivatives, or other ligands that can specifically bind to receptors on targeted cells. In one aspect, the targeting compound is a peptide such as, for example, an RGD peptide or bombesin peptide.

Any of the modified hyaluronans described herein can be the pharmaceutically-acceptable salt or ester thereof. Depending upon the reaction conditions, the hyaluronan portion and/or the hydrophobic polypeptide of the modified hyaluronan can be converted to the pharmaceutically-acceptable salt or ester. In one aspect, pharmaceutically-acceptable salts are prepared by treating the free acid with an appropriate amount of a pharmaceutically-acceptable base. Representative pharmaceutically-acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C. such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

In another aspect, if the modified hyaluronan possesses a basic group, it can be protonated with an acid such as, for example, HCl, HBr, or H₂SO₄, to produce the cationic salt. In one aspect, the reaction of the compound with the acid or base is conducted in water, alone or in combination with an inert, water-miscible organic solvent, at a temperature of from about 0° C. to about 100° C. such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

Ester derivatives are typically prepared as precursors to the acid form of the compounds. Generally, these derivatives will be lower alkyl esters such as methyl, ethyl, and the like. Amide derivatives —(CO)NH₂, —(CO)NHR and —(CO)NR₂, where R is an alkyl group defined above, can be prepared by reaction of the carboxylic acid-containing compound with ammonia or a substituted amine.

In one aspect, any of the modified hyaluronans described above can be used a pharmaceutical. In another aspect, any of the compounds produced by the methods described above can include or be combined with at least one pharmaceutically-acceptable compound. The resulting pharmaceutical composition can provide a system for sustained, continuous delivery of drugs and other biologically-active agents to tissues adjacent to or distant from the application site. The biologically-active agent is capable of providing a local or systemic biological, physiological or therapeutic effect in the biological system to which it is applied. For example, the agent can act to control infection or inflammation, enhance cell growth and tissue regeneration, control tumor growth, act as an analgesic, promote anti-cell attachment, and enhance bone growth, among other functions. Additionally, any of the compounds described herein can contain combinations of two or more pharmaceutically-acceptable compounds.

In one aspect, the pharmaceutically-acceptable compounds can include substances capable of preventing an infection systemically in the biological system or locally at the defect site, as for example, anti-inflammatory agents such as, but not limited to, pilocarpine, hydrocortisone, prednisolone, cortisone, diclofenac sodium, indomethacin, 6∝-methyl-prednisolone, corticosterone, dexamethasone, prednisone, and the like; antibacterial agents including, but not limited to, penicillin, cephalosporins, bacitracin, tetracycline, doxycycline, gentamycin, chloroquine, vidarabine, and the like; analgesic agents including, but not limited to, salicylic acid, acetaminophen, ibuprofen, naproxen, piroxicam, flurbiprofen, morphine, and the like; local anesthetics including, but not limited to, cocaine, lidocaine, benzocaine, and the like; immunogens (vaccines) for stimulating antibodies against hepatitis, influenza, measles, rubella, tetanus, polio, rabies, and the like; peptides including, but not limited to, leuprolide acetate (an LH-RH agonist), nafarelin, and the like. All compounds are commercially available.

Additionally, a substance or metabolic precursor which is capable of promoting growth and survival of cells and tissues or augmenting the functioning of cells is useful, as for example, a nerve growth promoting substance such as a ganglioside, a nerve growth factor, and the like; a hard or soft tissue growth promoting agent such as fibronectin (FN), human growth hormone (HGH), a colony stimulating factor, bone morphogenic protein, platelet-derived growth factor (PDGF), insulin-derived growth factor (IGF-I, IGF-II), transforming growth factor-alpha (TGF-alpha), transforming growth factor-beta (TGF-beta), epidermal growth factor (EGF), fibroblast growth factor (FGF), interleukin-1 (IL-1), vascular endothelial growth factor (VEGF) and keratinocyte growth factor (KGF), dried bone material, and the like; and antineoplastic agents such as methotrexate, 5-fluorouracil, adriamycin, vinblastine, cisplatin, tumor-specific antibodies conjugated to toxins, tumor necrosis factor, and the like.

Other useful substances include hormones such as progesterone, testosterone, and follicle stimulating hormone (FSH) (birth control, fertility-enhancement), insulin, and the like; antihistamines such as diphenhydramine, and the like; cardiovascular agents such as papaverine, streptokinase and the like; anti-ulcer agents such as isopropamide iodide, and the like; bronchodilators such as metaproternal sulfate, aminophylline, and the like; vasodilators such as theophylline, niacin, minoxidil, and the like; central nervous system agents such as tranquilizer, B-adrenergic blocking agent, dopamine, and the like; antipsychotic agents such as risperidone, narcotic antagonists such as naltrexone, naloxone, buprenorphine; and other like substances. All compounds are commercially available.

The pharmaceutical compositions can be prepared using techniques known in the art. In one aspect, the composition is prepared by admixing the modified hyaluronan with a pharmaceutically-acceptable compound. The term “admixing” is defined as mixing the two components together so that there is no chemical reaction or physical interaction. The term “admixing” also includes the chemical reaction or physical interaction between the compound and the pharmaceutically-acceptable compound.

It will be appreciated that the actual preferred amounts of active compound in a specified case will vary according to the specific compound being utilized, the particular compositions formulated, the mode of application, and the particular situs and subject being treated. Dosages for a given host can be determined using conventional considerations, e.g. by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physicians Desk Reference, Barnhart Publishing (1999).

Pharmaceutical compositions described herein can be formulated in any excipient the biological system or entity can tolerate. Examples of such excipients include, but are not limited to, water, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity-enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol. In one aspect, the compounds described herein are admixed with a non-FDA approved delivery device such as, for example, sunscreen or a nutraceutical.

Pharmaceutical carriers are known to those skilled in the art. These most typically would be standard carriers for administration to humans, including solutions such as sterile water, saline, and buffered solutions at physiological pH.

Molecules intended for pharmaceutical delivery can be formulated in a pharmaceutical composition. Pharmaceutical compositions can include carriers, thickeners, diluents, buffers, preservatives, surface-active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally).

Preparations for administration include sterile aqueous or non-aqueous solutions, suspensions, and emulsions. Examples of non-aqueous carriers include water, alcoholic/aqueous solutions, emulsions or suspensions, including saline and buffered media. Parenteral vehicles, if needed for collateral use of the disclosed compositions and methods, include sodium chloride solution, Ringer's dextrose, dextrose and sodium chloride, lactated Ringer's, or fixed oils. Intravenous vehicles, if needed for collateral use of the disclosed compositions and methods, include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Dosing is dependent on severity and responsiveness of the condition to be treated, but will normally be one or more doses per day, with course of treatment lasting from several days to several months or until one of ordinary skill in the art determines the delivery should cease. Persons of ordinary skill can easily determine optimum dosages, dosing methodologies and repetition rates.

In one aspect, any of the pharmaceutical compositions can include living cells. Examples of living cells include, but are not limited to, fibroblasts, hepatocytes, chondrocytes, stem cells, bone marrow, muscle cells, cardiac myocytes, neuronal cells, or pancreatic islet cells.

### II. APPLICATIONS

The modified hyaluronans described herein have numerous biological and medical applications. In particular, the modified hyaluronan is an extrudable, viscous material that can quickly reform as a gel once in the body. For example, it can be drawn into a syringe and extruded through a needle tip. In one aspect, the modified hyaluronan has an elastic modulus G′ in excess of 100 Pa, and a low-shear-rate viscosity in excess of 10,000 Pa-s. By contrast, aqueous solutions of unmodified hyaluronan of the same backbone molecular weight at the same solution concentration have G′ values that are too small to measure (see Examples). Additionally, moderate levels of shear stress (e.g., 10 Pa) are sufficient to convert the modified hyaluronan hydrogel into a low viscosity fluid, and that the gel reforms after removal of shear stress. Not wishing to be bound by theory, it is believed the breakdown of hydrophobic interactions between the hydrophobic polypeptide chains under shear is responsible for producing a viscous material. As shown in the Examples, shear thinning was observed at very low shear rates. Thus, in one aspect, the modified hyaluronan can be injected into the body and serve as an associative thickener (i.e., viscosupplement) of the synovial fluid. For example, the modified hyaluronan can be injected into a joint such as the knee using syringes where it would be subjected to very high shear rates.

By increasing the viscosity of synovial fluid, the modified hyaluronan can reduce friction in a joint of a subject. Thus, in one aspect, the modified hyaluronan can treat a subject with arthritis or help prevent the onset of arthritis. For example, the modified hyaluronan can treat a joint with articular cartilage exhibiting degeneration caused by osteoarthritis by contacting the joint with the modified hyaluronan. The joint can include knees, shoulders and sacroiliac, coxofemoral, ankles, elbows, interphalangeal, and wrists.

Also described here are methods for the treatment or prevention of intervertebral disc degeneration, where the method comprises administering the modified hyaluronan into an intervertebral disc. Back pain is the second most common ailment complained about in doctors' offices after the common cold and is responsible for some 100 million lost days of work annually in the United States alone. A major proportion of these back injuries result from disorders of the intervertebral discs in the spine. Although the exact pathogenesis of many intervertebral disc disorders is unknown, disorders such as degenerative disc disease are generally mechanically induced and biologically mediated.

Unfortunately, current treatment of intervertebral disc disorders, including IVD degeneration, has been limited to only a few courses of action, the most common of these being spinal surgery. Even though in some cases spinal surgery achieves over 90% good to excellent results, the pathological IVD, with time, continues to undergo degeneration and significant disability may still result. Furthermore, although surgical techniques such as lumbar spinal fusion have a high success rate if performed on patients with deformities or documented instabilities such as spondylolisthesis and scoliosis, the outcome of surgical procedures for low back pain without radiculopathy is unpredictable. The modified hyaluronans described herein provide an attractive alternative to the invasive techniques currently used.

In another aspect, the modified hyaluronan can minimize an imperfection in a subject's skin. In one aspect, the method involves injecting the modified hyaluronan in the skin of the subject at or near the imperfection. Examples of imperfections include, but are not limited to, a wrinkle, a skin fold, a scar, atrophied skin, and sunken skin. In one aspect, the modified hyaluronan can correct tissue prolapse and/or tissue atrophy (or loss) by administering injections of the modified hyaluronan into the deep part of the skin (i.e., deep fat or just above the bone). In another aspect, the modified hyaluronan can be used as a non-surgical cosmetic implant. For example, the implant can be a cheek or chin implant. The methods disclosed in U.S. Pat. No. 7,491,709, which are incorporated by reference, can be used herein for the application of modified hyaluronans in cosmetic applications.

The modified hyaluronans described herein can deliver at least one pharmaceutically-acceptable compound to a patient in need of such delivery. In one aspect, the method involves contacting at least one tissue capable of receiving the pharmaceutically-acceptable compound with one or more modified hyaluronans described herein. The modified hyaluronans described herein can be used as a carrier for a wide variety of releasable biologically active substances having curative or therapeutic value for human or non-human animals. Many of these substances that can be carried by the compound are discussed above. Included among biologically active materials which are suitable for incorporation into the gels of the invention are therapeutic drugs, e.g., anti-inflammatory agents, anti-pyretic agents, steroidal and non-steroidal drugs for anti-inflammatory use, hormones, growth factors, contraceptive agents, antivirals, antibacterials, antifungals, analgesics, hypnotics, sedatives, tranquilizers, anti-convulsants, muscle relaxants, local anesthetics, antispasmodics, antiulcer drugs, peptidic agonists, sympathiomimetic agents, cardiovascular agents, antitumor agents, oligonucleotides and their analogues and so forth. A biologically active substance is added in pharmaceutically active amounts.

In one aspect, the modified hyaluronans described herein can be used for the delivery of living cells to a subject.

In one aspect, the modified hyaluronans can be used for the delivery of growth factors and molecules related to growth factors. For example the growth factors can be a nerve growth promoting substance such as a ganglioside, a nerve growth factor, and the like; a hard or soft tissue growth promoting agent such as fibronectin (FN), human growth hormone (HGH), a colony stimulating factor, bone morphogenic protein, platelet-derived growth factor (PDGF), insulin-derived growth factor (IGF-I, IGF-II), transforming growth factor-alpha (TGF-alpha), transforming growth factor-beta (TGF-beta), epidermal growth factor (EGF), fibroblast growth factor (FGF), interleukin-1 (IL-1). In one aspect, the growth factors are bFGF, TGF-β, vascular endothelial growth factor (VEGF) and keratinocyte growth factor (KGF).

Also described herein are methods for improving wound healing in a subject in need of such improvement by contacting any of the a wound of a subject in need of wound healing improvement with the modified hyaluronan. The compositions described herein can be placed directly in or on any biological system without purification as it is composed of biocompatible materials. Examples of sites the modified hyaluronans can be placed include, but not limited to, soft tissue such as muscle or fat; hard tissue such as bone or cartilage; areas of tissue regeneration; a void space such as periodontal pocket; surgical incision or other formed pocket or cavity; a natural cavity such as the oral, vaginal, rectal or nasal cavities, the cul-de-sac of the eye, and the like; the peritoneal cavity and organs contained within, and other sites into or onto which the compounds can be placed including a skin surface defect such as a cut, scrape or burn area. It is contemplated that the tissue can be damaged due to injury or a degenerative condition or, in the alternative, the compounds and compositions described herein can be applied to undamaged tissue to prevent injury to the tissue. The present modified hyaluronans can be biodegradeable and naturally occurring enzymes will act to degrade them over time. Components of the compound can be “bioabsorbable” in that the components of the compound will be broken down and absorbed within the biological system, for example, by a cell, tissue and the like.

The modified hyaluronans can be used for treating a wide variety of tissue defects in an animal, for example, a tissue with a void such as a periodontal pocket, a shallow or deep cutaneous wound, a surgical incision, a bone or cartilage defect, and the like. The modified hyaluronan can be applied to a defect in bone tissue such as a fracture in an arm or leg bone, a defect in a tooth, a cartilage defect in the joint, ear, nose, or throat, and the like. The hydrogel film composed of the compound described herein can also function as a barrier system for guided tissue regeneration by providing a surface on or through which the cells can grow.

The modified hyaluronan can be delivered onto cells, tissues, and/or organs, for example, by injection, spraying, squirting, brushing, painting, coating, and the like. Delivery can also be via a cannula, catheter, syringe with or without a needle, pressure applicator, pump, and the like. The compound can be applied onto a tissue in the form of a film, for example, to provide a film dressing on the surface of the tissue, and/or to adhere to a tissue to another tissue or hydrogel film, among other applications.

In one aspect, the modified hyaluronan is administered via injection. For many clinical uses, when the modified hyaluronan is in the form of a hydrogel film, injectable hydrogels are preferred for three main reasons. First, an injectable hydrogel could be formed into any desired shape at the site of injury. Second, the modified hyaluronan would adhere to the tissue during gel formation, and the resulting mechanical interlocking arising from surface microroughness would strengthen the tissue-hydrogel interface. Third, introduction of the modified hyaluronan could be accomplished using needle or by laparoscopic methods, thereby minimizing the invasiveness of the surgical technique.

The modified hyaluronans described herein can be used to treat periodontal disease, gingival tissue overlying the root of the tooth can be excised to form an envelope or pocket, and the composition delivered into the pocket and against the exposed root. The modified hyaluronans can also be delivered to a tooth defect by making an incision through the gingival tissue to expose the root, and then applying the material through the incision onto the root surface by placing, brushing, squirting, or other means.

The modified hyaluronans described herein can be applied to an implantable device such as a suture, clamps, prosthesis, catheter, stents, metal screw, bone plate, pin, a bandage such as gauze, and the like, to enhance the compatibility and/or performance or function of an implantable device with a body tissue in an implant site. The modified hyaluronans can be used to coat the implantable device. For example, the modified hyaluronans could be used to coat the rough surface of an implantable device to enhance the compatibility of the device by providing a biocompatible smooth surface that reduces the occurrence of abrasions from the contact of rough edges with the adjacent tissue. The modified hyaluronans can also be used to enhance the performance or function of an implantable device. For example, when the modified hyaluronan is a hydrogel film, the hydrogel film can be applied to a gauze bandage to enhance its compatibility or adhesion with the tissue to which it is applied. The hydrogel film can also be applied around a device such as a catheter or colostomy that is inserted through an incision into the body to help secure the catheter/colostomy in place and/or to fill the void between the device and tissue and form a tight seal to reduce bacterial infection and loss of body fluid. In one aspect, the modified hyaluronans can be coated onto metal stents (titanium, nickel, gold, etc.) used in angioplasty (atherosclerosis) and prevent restenosis by preventing scar tissue formation. In another aspect, the modified hyaluronans described herein can be used to coat metal joints.

It is understood that any given particular aspect of the disclosed compositions and methods can be easily compared to the specific examples and embodiments disclosed herein, including the non-polysaccharide based reagents discussed in the Examples. By performing such a comparison, the relative efficacy of each particular embodiment can be easily determined. Particularly preferred compositions and methods are disclosed in the Examples herein, and it is understood that these compositions and methods, while not necessarily limiting, can be performed with any of the compositions and methods disclosed herein.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### I. Materials and Methods

Sodium hyaluronate of four different average molecular weights (67 k, 74 k, 132 k, and 1.5M Daltons, see Table 1) were purchased from Lifecore Biomedical (Chaska, Minn.). The 74 kDa and 132 kDa samples were used in the synthesis of leucine-modified HA. Results for solutions of these leucine-modified HA derivatives are compared below to results for solutions of unmodified HA at 132 kDa and 67 kDa, due to the unavailability of additional amounts of 74 kDa HA at Lifecore Biomedical. Solutions of various concentrations in distilled deionized water containing 0.1 M NaCl were prepared by gentle mixing on a lab rocker with mixing times between a few hours to days depending on the concentration and molecular weight. The solutions were stored in the refrigerator at 0° C. when not in use.

### Synthesis of Monodisperse Poly(Leucine) Chains

Using DMF as the solvent, at room temperature leucine was reacted with N-Carboxy Anhydride (NCA) for initiation of the polymerization reaction. The addition reaction involves ring opening of NCA and elimination of one CO₂ molecule for each unit of leucine added to the chain. The polymerization proceeds by an anionic primary amine mechanism resulting in polypeptides with a very narrow molecular weight distribution (FIG. 1).

Grafting Poly(Leucine) Branches onto HA Chains

HA of two different molecular weights were grafted with polypeptide chains about 10 leucine units long. Peptide-modified HA with 74 k Da backbone HA is designated sample B1, and the peptide-modified HA with the 132 kDa backbone HA is sample A5. Michael addition reaction was used to modify linear HA chains with polypeptide branches. First, HA was functionalized with tertbutyl ammonium (HA-TBA). HA-TBA was then esterified with acrylolchloride to introduce a vinyl group on the hydroxyl group on glucosamine in the HA repeat unit. Michael addition reaction was then done with HA-TBA vinyl groups and the primary amine group on the poly(leucine) (FIG. 2).

TABLE 1
Properties of biopolymers used in this study. Peptide-modified HA with 74 kDa backbone is
referred to as B1 and peptide-modified HA with 132 kDa backbone is referred to as A5.

| | | | | | | | Grafting ratio |
|---|---|---|---|---|---|---|---|
| | | | Overlap | R_{H} (nm) | R_{H} (nm) | R_{H} (nm) | (number of |
| | Weight-Average | Intrinsic | concentration | (estimated | (measured | (measured | polypeptide |
| | Molecular | Viscosity | c* ≈ 1/[η] | from intrinsic | using | using PD | chains per |
| Material | weight (g/mol) | [η] (dl/g) | (mg/ml) | viscosity) | Nanosizer) | Expert) | repeat unit) |
| Unmodified | 67,000 | 2.31 | 4.33 | 13.488 | 11.28 | — | — |
| HA | 132,000 | 3.42 | 2.92 | 19.46 | 15.37 | 15.9 | |
| | 1,500,000 | 20.66 | 0.48 | 78.91 | — | — | |
| Leucine- | 74 k Da | 1.72 | 5.8 | 18.86^{a} | 367.1 | — | 0.015 |
| modified | backbone (B1) | | | | | | |
| HA | 132 k Da | 0.76 | 13.15 | 18.12^{a} | 851.56 | 125.7 | 0.037 |
| | backbone (A5) | | | | | | |
| Poly-L- | 2000 | — | — | — | — | — | — |
| Leucine | | | | | | | |

^{a}Calculated assuming no aggregation

### Viscometry

A Cannon-Manning semi-micro viscometer (size 50) with an efflux volume of 0.2-0.3 ml was used to measure relative viscosities of very dilute solutions, in order to obtain their intrinsic viscosities. The capillary diameter of the viscometer is 0.013 cm and a shear rate of ˜1200 s⁻¹ was calculated for this viscometer. A constant temperature of 24° C. was maintained using a water bath.

### Rheometry

A constant stress rheometer AR550 (TA instruments) was used for all rheological measurements, with a stainless steel cone and plate geometry (cone angle 2 degrees, radius 20 mm). The temperature was maintained at 23° C. which is slightly less than the room temperature, in order to help prevent evaporation.

### Dynamic Light Scattering (DLS)

DLS was performed using a Nanosizer (Malvern Instruments) at 24° C. to obtain the hydrodynamic radii of the various samples or by PD Expert Instrument (Precision Detectors Inc., Massachusetts) (Table 1).

### Pendant Drop Surface Tension Measurements

Surface tension experiments were conducted on the modified and unmodified HA solutions using a pendant drop apparatus developed in-house. Gas tight glass syringes (250 μl, Hamilton Inc.) with flat-tip stainless steel needles (14 gauge) were used to form pendant drops of actin solutions in a transparent environmental chamber. Drop images were captured at various times after drop formation using a CCD camera with image analysis to extract the interfacial coordinates. The surface tension and the drop area were obtained as functions of time by fitting each drop shape to an axisymmetric form of the Young-Laplace equation of capillarity, derived specifically for pendant shapes from a force-balance over the suspended drop. Drop density was assumed to equal that of water; all measurements were performed at room temperature (24° C.±1° C.).

### II. Results and Discussion

### Dynamic Light Scattering

R_{H} values of the various polymer samples from DLS were determined (Table 1). When illuminated with a monochromatic light source with a wavelength having a comparable size, polymer chains in a dilute solution scatter light. Due to Brownian motion of these suspended chains, the scattering intensity changes with time. An analysis of the fluctuations in the scattered light intensity gives the diffusion coefficient (D) of the particles in the medium. Using the Stokes-Einstein relation, this diffusivity is then used to arrive at the hydrodynamic radius (R_{H}) of the polymer chains. Hydrodynamic radius is the radius of an imaginary hard sphere with diffusivity equal to the diffusivity of the polymer chain in solution. In reality, the polymer chains would be non-spherical, dynamic i.e constantly changing shape and hydrated. Hence, R_{H} takes into account both the viscosity of the medium and the shape of the polymer chain in this medium.

D=kT/f=kT/6πηR_{H} (1)

where k=Boltzman constant,
T=temperature
η=solvent viscosity
f=frictional coefficient for a hard sphere in a viscous medium.

As shown in Table 1, the R_{H} values of the unmodified HA samples measured by both DLS instruments are in good agreement, but this was not the case with peptide-modified sample A5. For the peptide-modified sample, the R_{H} values obtained from DLS are so large that self-association of the molecules into aggregates almost certainly must have occurred.

### Viscometry

Very dilute solutions of the various modified and unmodified samples were prepared in 0.1M NaCl. Their flow times (t) were measured using the Cannon-Manning semi-micro viscometer from which their specific viscosities (ηₛₚ) were calculated.

ηₛₚ=(t−tₛ)/tₛ=ηᵣ−1 (2)

where t=solution flow time; tₛ=solvent flow time; ηᵣ=relative viscosity

Intrinsic viscosities ([η]) of the HA samples were then estimated from these measurements. IV is a measure of the fractional contribution of the polymer to solution viscosity, extrapolated to infinite dilution.

[η]=Lt(c→0)(η−ηₛ)/ηₛc (3)

where η is the solution viscosity; ηₛ is the solvent viscosity and c is the concentration of the solutions.

It is also a measure of the ratio of molecular mass of the coil to volume pervaded by the polymer coil in solvent and is estimated to be the inverse of overlap concentration, c*.

The minimum concentration at which a rapid rise in solution viscosity is observed corresponds to coil overlap and is known as the overlap concentration (c*). It can be estimated as the concentration at which the product of the number of coils per unit volume (v) and volume (R_{g}³) pervaded by a single coil is approximately unity.

c*=v*M/N_{A}=R_{g}⁻³M/N_{A} (4)

where M is the molecular weight of the polymer coil, N_{A} is the Avogadro's number and R_{g} is the radius of gyration of the polymer coil.

Hence, the overlap concentration (c*) depends on the coil molecular weight as well as the volume pervaded by the coil in solution. Below c*, the solution is considered to be in the dilute regime and polymer coils do not have any influence on each other's behavior. However, for high molecular weight polymers such as physiological HA, occasional coil overlap may occur even below experimentally measured c*.

Hence true dilute solution properties can be obtained only by extrapolating the low concentration values to zero concentration. IV is a dilute solution property. Larger polymer chains would have a higher intrinsic viscosity and overlap at lower concentrations.

[η]≈1/c* (5)

Using the intrinsic viscosities, the R_{H} values were estimated using the following equation:

R_{H}³=[η]M.3/(10πN_{A}) (6)

Note that this equation assumes that the molar mass M is known, which may not be the case for hydrophobically-modified HA if aggregation occurs. For unmodified HA samples, good agreement was found between R_{H} estimated by this method and DLS. However, the peptide-modified HA samples showed significantly higher values in DLS than that estimated using the intrinsic viscosity (see Table 1). The R_{H} values from DLS are so large that molecular aggregates must have been present, and the resulting polydispersity may have distorted the mean coil size calculated by the DLS software. Alternatively, it is possible that no aggregates were present in the IV measurements due to the high shear rates in the viscometer (˜1200 s⁻¹ at the wall) compared to the nearly zero shear rates in DLS, indicating a shear dependent behavior in modified HA.

### Surface Tension Measurements

Surprisingly, even though the leucine-modified HA biopolymers should be amphiphilic, the equilibrium surface tension value at 0.05 mg/ml was essentially identical for native HA 67 kDa and the peptide-modified sample B1 (62 dyne/cm). This may indicate that B1 forms association complexes, with the leucine branches segregated into the hydrophobic core. Such an association complex would not be expected to be surface active.

### Zero-Shear-Limiting Viscosity (η₀)

For a viscoelastic liquid, the zero-shear-rate-limiting viscosity ηₒ can be obtained either from linear oscillatory shear measurements at sufficiently low frequency, or from steady shear flow measurements at sufficiently low shear stress. However, if leucine-modified HA forms a long-lived physical network, then it may be experimentally impossible to perform measurements at sufficiently low frequency due to occurrence of sample evaporation. Therefore, for leucine-modified HA solutions, η₀ was obtained from steady-shear flow measurements at a constant shear stress 0.2 Pa, whereas for unmodified HA solutions, η₀ was obtained using dynamic oscillatory measurements, with a stress amplitude of 0.2 Pa and an angular frequency of 0.2 rad/s.

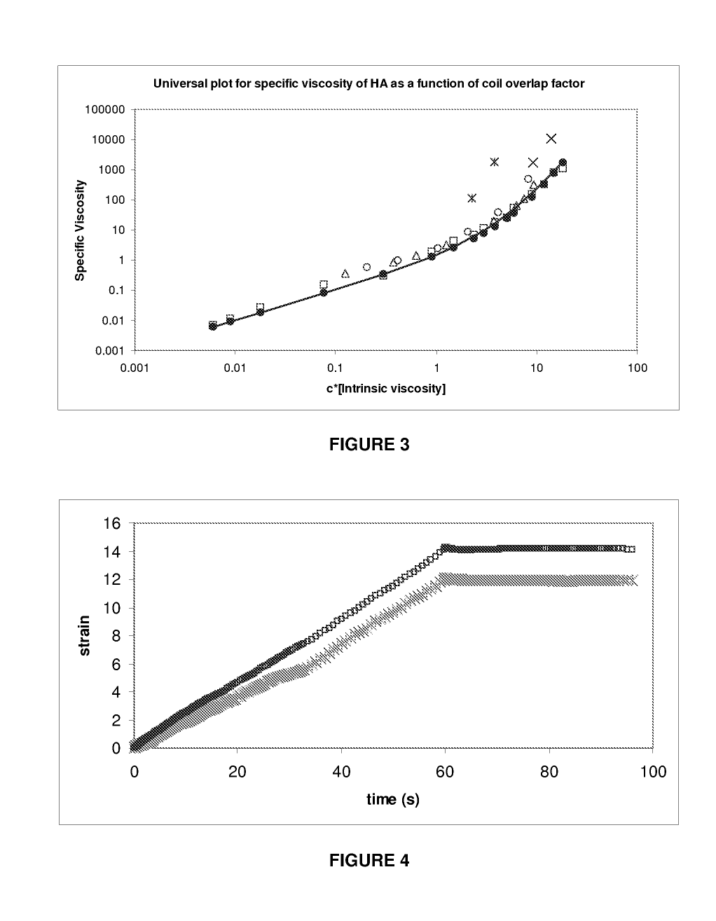

The specific viscosities of the various materials were plotted as a function of their overlap factor C/C* and compared to a universal plot developed for linear HA solutions under various aqueous conditions and other experimental results and from the literature (FIG. 3).

As seen in FIG. 3, the η₀ values for unmodified HA at all molecular weights and concentrations studied here are in good agreement with the universal correlation. However, peptide-modified HA samples A5 and B1 showed a significant enhancement of the zero-shear-rate-limiting viscosity, attributable to associative thickening. For peptide-modified HA sample B1, the enhancement of η₀ is very large (in excess of 10,000 Pa-s) at large overlap factors. At high concentrations, sample B1 may in fact be a weak solid with a yield stress value of less than 0.2 Pa. Even at the lowest shear stresses studied, the network structure may have been broken. Hence, the corresponding viscosity measured may be that of the broken network structure rather than the original undeformed structure.

### Creep-Recovery Behavior

Creep-recovery experiments were performed on the leucine-modified HA samples to study their viscoelastic nature. In a creep-recovery experiment, the sample is subjected to a constant stress for a given period of time during which the sample strain is measured, followed by removal of the stress. If the sample is viscoelastic, then a portion of the creep strain will be recovered after stress removal.

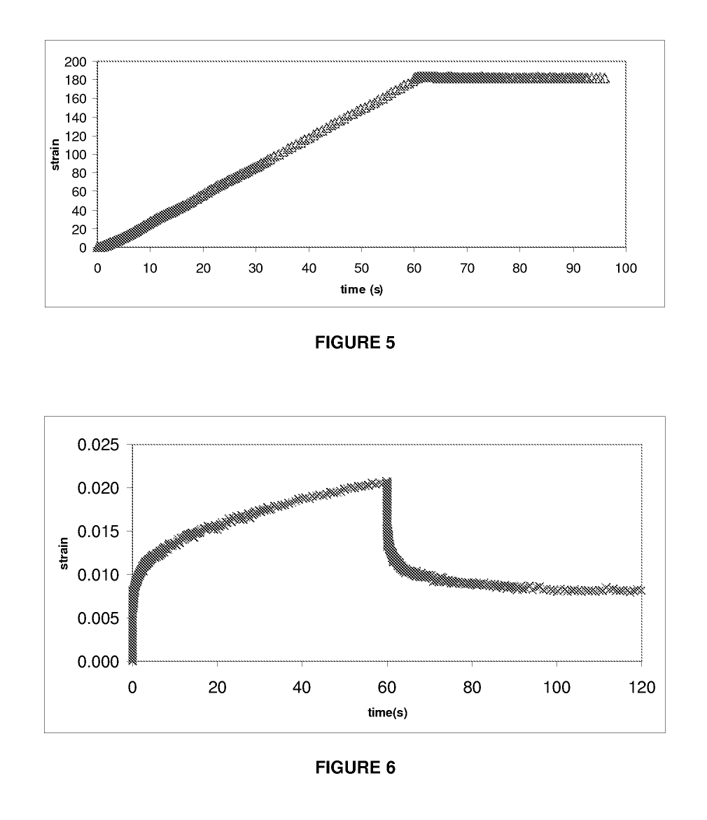

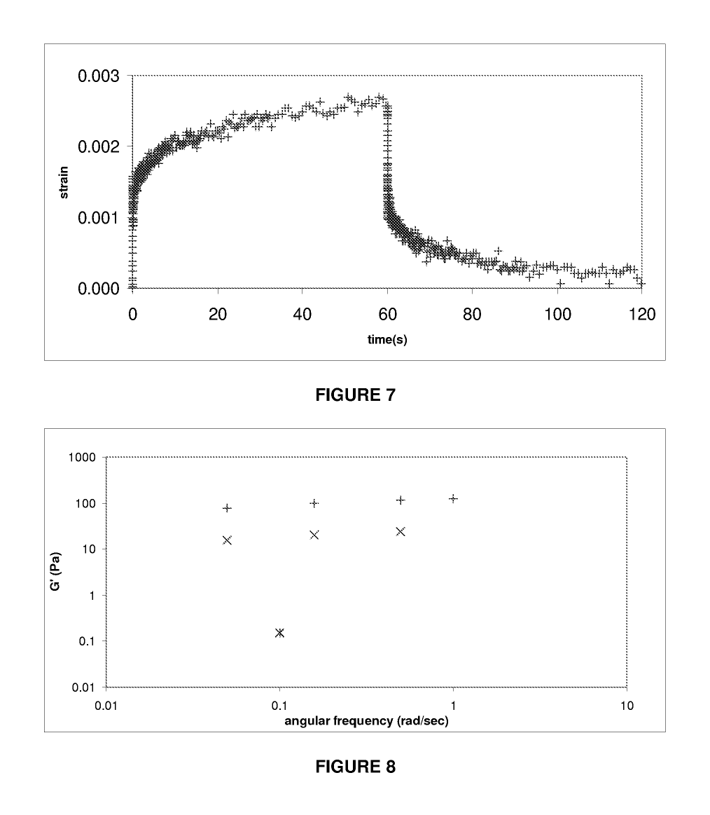

Creep recovery behavior of modified and unmodified HA is shown in FIGS. 4-7. Leucine-modified HA sample B1 shows clear evidence of viscoelastic behavior, possibly even that of a true gel at higher concentrations. On the other hand, leucine-modified HA sample A5 shows only viscous liquid response, even though it has a higher molecular weight and grafting ratio than B1. The viscoelastic behavior of B1 also showed concentration dependent behavior.

At higher concentrations (7.5%) the contribution of viscous flow to the total creep (σ₀t/η) was much lower and the material nearly recovered its original state upon removal of stress. The 7.5% B1 also showed a maximum strain which was more than 7 times lower than the 5% B1 sample under same stress.

At constant stress, a viscous liquid shows a linear increase in strain with time and a constant strain upon removal of stress. Whereas an elastic solid attains a constant strain and reverts completely to its original state upon removal of stress.

As shown in Table 1, the modified HA with 132 k backbone (A5) has a grafting ratio (number of polypeptide chains per repeat unit) of 0.037 or nearly 11 polypeptide chains per HA chain, which is much higher than the grafting ratio in B1 of 0.015 which is about 2.4 polypeptide chains per HA chain.

The probability of hydrophobic interactions and micelle formation in the modified HA samples would be higher with a higher grafting ratio. However, a higher grafting ratio could also lead to excessive intramolecular interactions leading to collapse of the chain.

Hence, A5 with a higher grafting ratio probably undergoes collapse. The significantly higher zero shear viscosity of A5 compared to unmodified HA with same backbone molecular weight may be due to a higher hydrodynamic radius rather than hydrophobic interactions as expected.

### Linear Oscillatory Shear Measurements

FIG. 8 shows the elastic modulus G′ for leucine-modified HA solutions as a function of frequency as obtained at a stress amplitude of 0.2 Pa. For sample B1, G′ is independent of frequency and greater than G″ down to the lowest frequencies that could be accessed without sample evaporation problems, 0.05 rad/sec. Since the lifetime of a physical network is given by the crossover frequency of G′ and G″, the lifetime of the B1 network is at least 20 s.

The storage modulus of the modified HA was much higher than unmodified HA of same backbone molecular weight, as expected. However, unexpectedly, B1 exhibits a significantly higher G′ value than A5 at the same concentration.

### Shear-Thinning/Thixotropy Measurements

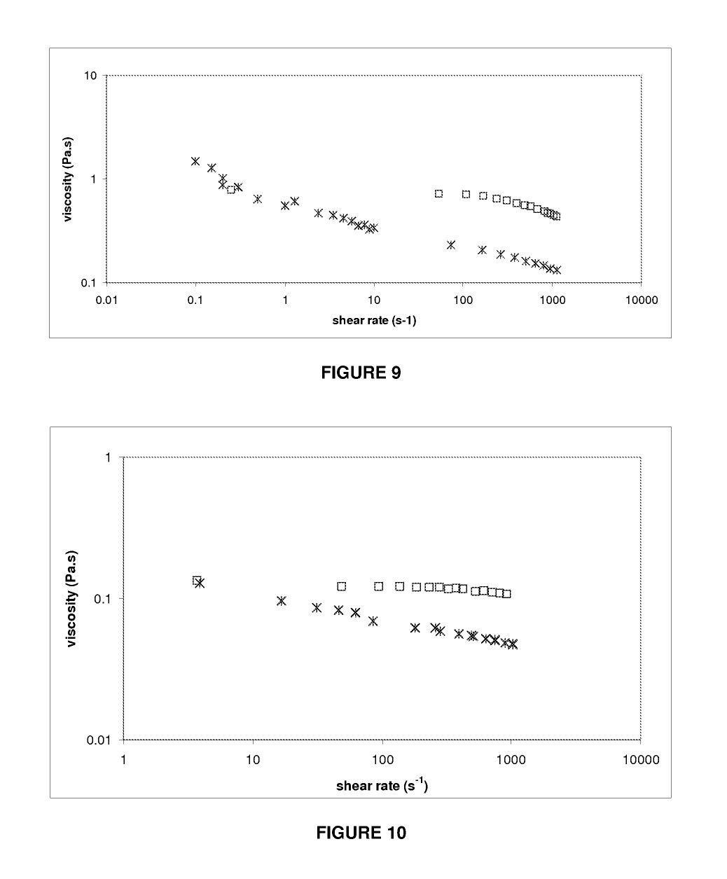

The steady state viscosities of A5 were measured in the shear rate range 0.1-1000 s⁻¹. Compared to the unmodified HA, the modified HA showed severe shear thinning. At a shear rate of ˜1000 s⁻¹, 3% and 5% A5 solutions showed 62% and 70% reduction, respectively, from their values at a low shear rate of 4 s⁻¹ (FIGS. 9 and 10).

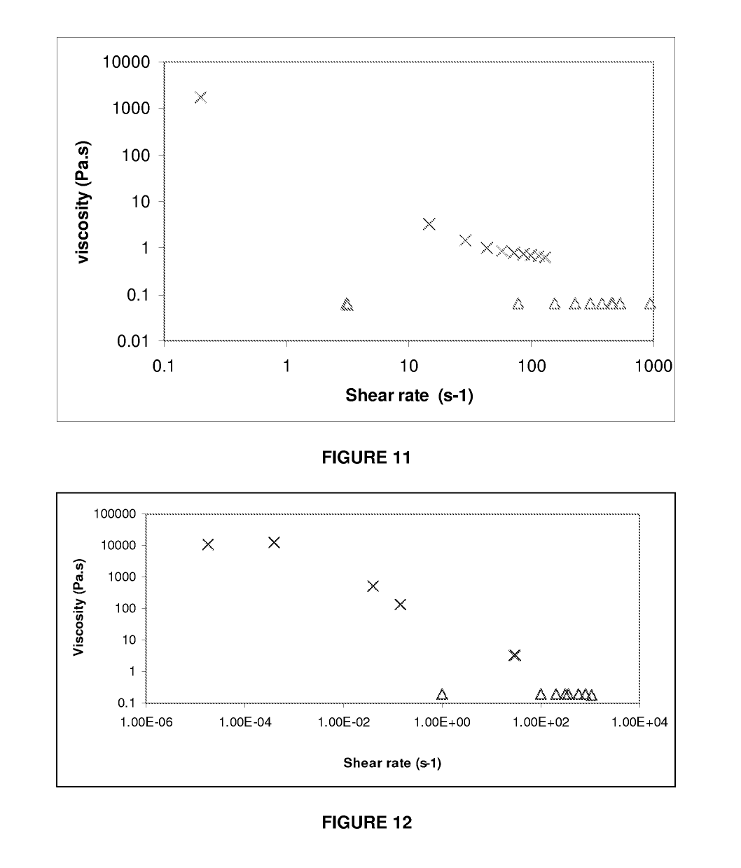

The 5% B1 sample showed a very severe shear thinning as shown in FIG. 11 below. Compared to its value at the lowest shear rate (0.2 s⁻¹) a 5% B1 solution showed nearly 100% reduction in viscosity at a shear rate of 15 s⁻¹ while unmodified HA (67 kDa) showed no shear thinning even up to a shear rate of 900 s⁻¹.

### Thixotropy and Network Recovery of 7.5 Wt % Poly(Leucine)-Modified Sample B1

FIG. 12 shows the results obtained for viscosity vs. shear rate of leucine-modified sample B1 at 7.5 wt %. The decrease in viscosity is too severe to be interpreted as normal polymer shear-thinning behavior, thus thixotropic breakdown of the network must be involved. Network recovery of 7.5% B1 was studied using stopped-flow experiments. In these experiments, after each measurement of the steady flow viscosity, linear oscillatory shear measurements (0.1 rad/sec, 0.2 Pa) of the network elastic modulus G′ were performed as quickly as possible to probe the breakdown of network structure with increasing shear rate. The time between consequent measurements is carefully noted in order to obtain the time required for the sample to recover its original properties after network deformation.

The time between consequent measurements was on an average 1±0.1 minutes. After each value of the shear rate shown in FIG. 12, the measured value of G′ was determined to be equal to that of the fresh sample. Hence for 7.5 wt % B1, we found a network recovery time of at most 1 minute.

### Surface Tensions at Low Concentrations of Modified and Unmodified HA Samples

Surface tensions were measured at a temperature of 24° C.±1° C. using the pendant drop technique. The first data point was collected at about 10 seconds after drop formation. Already after 10 s the surface tension had decreased from the pure water value of 70 dyne/cm, indicating adsorption of some molecules with large diffusivities. The tension continues to decrease with time as more molecules adsorb at the air/buffer interface, as shown in FIGS. 10 and 11.

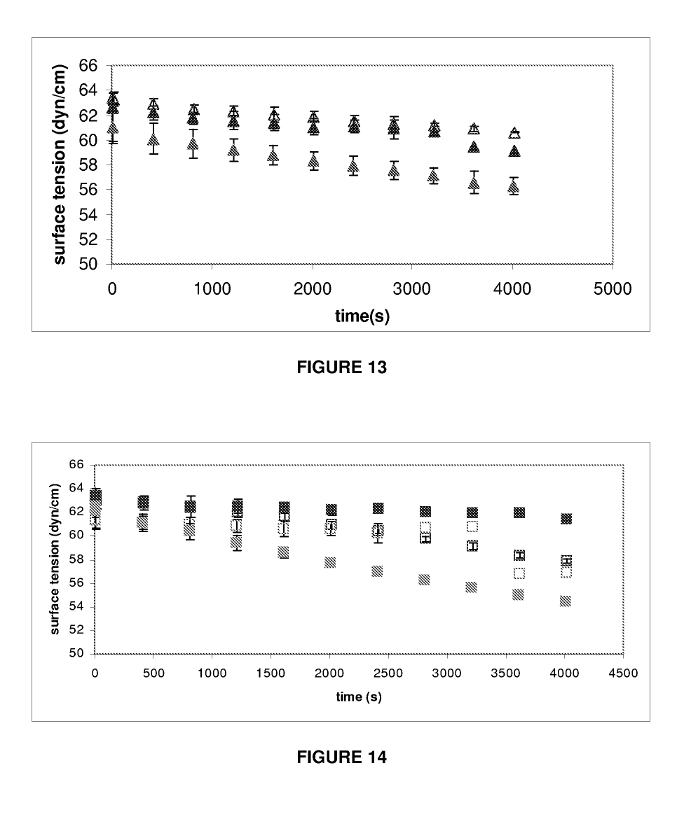

FIG. 13 shows the surface tension of 67 k Da unmodified HA and B1 at 0.05 mg/ml concentration. As expected, B1 shows a greater decrease in surface tension over time due to a higher hydrophobicity due to the presence of hydrophobic poly(leucine) side chains. The surface tension of a 1 mg/ml B1 solution was also found to be lower than the 0.05 mg/ml solution (FIG. 13).

FIG. 14 shows the surface tension behavior of unmodified 132 k Da HA and A5 solutions at 0.05 mg/ml and 1 mg/ml concentrations. At 1 mg/ml concentration, the decrease in surface tension of A5 is higher than the 132 k Da unmodified HA, indicating a higher hydrophobicity of A5 and a greater affinity for the interface with air. However, A5 at a lower concentration showed a less hydrophobic behavior with a surface tension much higher than for unmodified 132 kDa HA at the same concentration.

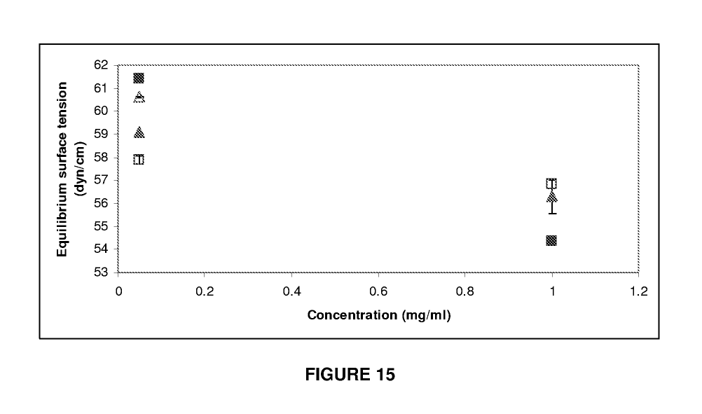

FIG. 15 shows the equilibrium surface tensions of modified and unmodified HA as a function of concentration. The surface tension values at ˜4000 seconds were taken as the equilibrium values. Both modified and unmodified HA samples show a decrease in equilibrium surface tension at higher concentrations. However, at the lower concentration the equilibrium surface tension of A5 is greater than unmodified 132 k Da HA. The same is not true for B1 and 67 k Da unmodified HA. This could probably be because of a higher number of poly(leucine) branches per chain in A5, which at this concentration tend to show intramolecular aggregation.

### III. CONCLUSIONS

The rheological properties of HA modified with hydrophobic poly(leucine) side chains for use as a potential viscosupplement were investigated.

For unmodified HA at all concentrations good agreement was obtained between experimental and literature values of zero shear viscosities.

At higher concentrations, modified HA (both 67 k Da and 132 k Da backbone) showed enhanced zero shear viscosities, which were attributed to associative thickening due to hydrophobic interactions between poly(leucine) side chains.

The intrinsic viscosities of all the HA samples were measured using a semi-microviscometer in which the shear rate during capillary flow is nearly 1200 s⁻¹. R_{H} of the materials was estimated from the IV measurements. The estimated R_{H} values of the unmodified HA were in excellent agreement with the values obtained using DLS. However, for the unmodified HA, DLS measurements showed an R_{H} several orders of magnitudes higher than the R_{H} estimated from intrinsic viscosity measurements. This was attributed to shear thinning during capillary flow in the viscometer.

This theory was further corroborated when, compared to the unmodified HA of 132 k Da molecular weight, a high degree of shear thinning (60-70% drop) was observed for A5 (3% and 5% concentrations) at shear rates as low as 4 s⁻¹. At a 5% concentration, B1 showed even higher shear thinning with almost a 100% drop in viscosity at a shear rate of 15 s⁻¹. No shear thinning was observed in unmodified HA of 67 k Da backbone even at higher concentrations of 7.5% up to shear rates of 1000 s⁻¹.

Hence, the effect of shear thinning was attributed to the breakdown of hydrophobic interactions between the poly(leucine) chains under shear.

Shear thinning was observed at very low shear rates. This would be advantageous for viscosupplements during injection into the knee using syringes where they would be subjected to very high shear rates.

Another advantage of the modified HA was that quick network recovery was observed after deformation of the network structure by very high shear stresses. Network recovery of B1 was studied using flow-stop experiments. Less than 1±0.1 minute was required for the network properties to return to their original values. B1 showed viscoelastic liquid behavior and approached viscoelastic gel like behavior at higher concentrations with a phase angle of 10-15°. The elastic moduli of the modified HA were significantly higher than the corresponding unmodified HA at the same concentration. However, B1 showed a significantly higher value at the same concentration compared to A5, thus indicating that the associative interactions were stronger in B1 than A5. B1 also showed an increase in the elastic modulus with increasing angular frequencies.

The modified HA with a 67 k Da backbone showed creep-recovery behavior typical of viscoelastic liquids. At higher concentrations, the contribution of viscous flow to the creep was significantly lower. At the same concentrations unmodified HA and A5 showed creep-recovery typical of viscous liquids. This was attributed to the higher number of hydrophobes per chain in A5, which causes excessive hydrophobic interactions and leads to collapse of the chain into more compact coils.

This result was also supported by simulation studies. However, even the collapsed A5 chains showed a higher zero shear viscosity compared to unmodified 132 k Da HA, probably due to a higher R_{H}. B1 shows effects of associative thickening due to hydrophobic interactions between the poly(leucine) chains even with a lower grafting ratio.

Surface tension of both modified and unmodified HA at all molecular weights showed a concentration dependent behavior. A greater decrease in surface tension was observed at higher concentrations. As expected, A5 showed a lower surface tension than B1 at 1 mg/ml, probably due to a higher R_{H}. On the contrary, at a lower concentration of 0.05 mg/ml, the surface tension of A5 was higher than B1, probably indicating greater hydrophobic intramolecular interactions due to a higher grafting ratio in A5.

Hence, the grafting ratio plays an important role in the associative thickening of the modified HA. Further studies will have to be carried out to find the optimum grafting ratio for modification of HA. The rheological properties indicate good potential for use of modified HA as a viscosupplement.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

1. A modified hyaluronan or the pharmaceutically-acceptable salt or ester thereof, wherein the modified hyaluronan comprises at least one hydrophobic polypeptide covalently bonded to hyaluronan.

2. The modified hyaluronan of claim 1, wherein the hyaluronan has a molecular weight of 50,000 to 10,000,000.

3. The modified hyaluronan of claim 1, wherein the hydrophobic polypeptide has from 5 to 20 amino acid residues.

4. The modified hyaluronan of claim 1, wherein the hydrophobic polypeptide has a molecular weight from 600 to 3,000.

5. The modified hyaluronan of claim 1, wherein the hydrophobic polypeptide comprises diblock polypeptide comprising a hydrophilic block and a hydrophobic block, wherein the hydrophilic block is covalently attached to hyaluronan.

6. The modified hyaluronan of claim 1, wherein the hydrophobic polypeptide is isotactic poly(leucine), atactic poly(leucine), or a combination thereof.

7. The modified hyaluronan of claim 1, wherein the modified hyaluronan comprises a residue of formula I 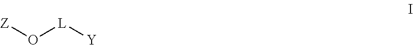
wherein
Z is a residue of hyaluronan;
L is a linker; and
Y is a residue of a hydrophobic polypeptide.

8. The modified hyaluronan of claim 1, wherein the modified hyaluronan comprises a residue of formula II 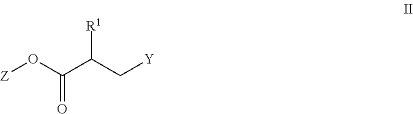
wherein
Z is a residue of hyaluronan;
R¹ is hydrogen or methyl; and
Y is a residue of a hydrophobic polypeptide.

9. The modified hyaluronan of claim 1, wherein R¹ is hydrogen and Y is poly(leucine).

10. The modified hyaluronan of claim 1, wherein the modified hyaluronan has a grafting ratio of 1% of the HA repeat units to 10% of the HA repeat units.

11. The modified hyaluronan of claim 1, wherein the modified hyaluronan is an extrudable composition.

12. The modified hyaluronan of claim 1, wherein the hydrophobic polypeptide comprises a targeting compound covalently bonded to the polypeptide.

13. A modified hyaluronan produced by the process comprising
a. reacting hyaluronan with a linker to produce a covalent bond between the linker and hyaluronan, wherein the linker possesses at least one group capable of reacting with an amino acid residue in a hydrophobic polypeptide to produce a covalent bond between the linker and the hydrophobic polypeptide; and
b. reacting the hyaluronan produced in step (a) with the hydrophobic polypeptide.

14. The modified hyaluronan of claim 13, wherein the linker possesses at least one group capable of undergoing a Michael addition with the hydrophobic polypeptide.

15. The modified hyaluronan of claim 13, wherein the linker comprises an acrylate or methacrylate.

16. The modified hyaluronan of claim 13, wherein the linker is acrylic acid, acrylic anhydride, or acrylolchloride.

17. A pharmaceutical composition comprising the modified hyaluronan in any of claim 1.

18. (canceled)

19. A method for treating or preventing arthritis in a subject comprising administering the modified hyaluronan in any of claim 1 to the subject.

20. The method of claim 19, wherein the modified hyaluronan is injected into the joint of a subject.

21. A method for reducing friction in a joint of a subject, the method comprising injecting the modified hyaluronan in any of claim 1 into the joint.

22. A method for increasing the viscosity of synovial fluid in a joint of a subject, the method comprising contacting the synovial fluid with the modified hyaluronan in any of claim 1.

23. A method for the treatment or prevention of intervertebral disc degeneration, the method comprising administering the modified hyaluronan in any of claim 1 into an intervertebral disc.

24. A method for minimizing an imperfection in a subject's skin, the method comprising injecting the modified hyaluronan in any of claim 1 in the skin of the subject at or near the imperfection.

25. The method of claim 24, wherein the imperfection is a wrinkle, a skin fold, a scar, atrophied skin, and sunken skin.

26. A method for improving wound healing in a subject in need of such improvement, comprising contacting the wound of the subject with the modified hyaluronan in any of claim 1.

27. A method for delivering at least one pharmaceutically-acceptable compound to a subject in need of such delivery, comprising administering to the subject the modified hyaluronan in any of claim 1.

28. A method for growing tissues, comprising contacting precursor cells with the modified hyaluronan in any claim 1.
